(19) United States
(12) Patent Application Publication
(10) Pub. No.: US 2023/0053587 A1
(43) Pub. Date: Feb. 23, 2023

(54) A MICRONEEDLE AND A FLUID CHANNEL SYSTEM FOR COLLECTING FLUID

(71) Applicant: Ascilion AB, Kista (SE)

(72) Inventors: Markus Renlund, Åkersberga (SE); Pelle Rangsten, Storvreta (SE); Mikael Hillmering, Sollentuna (SE)

(73) Assignee: Ascilion AB, Kista (SE)

(21) Appl. No.: 17/797,377

(22) PCT Filed: Feb. 10, 2021
(86) PCT No.: PCT/SE2021/050098

§ 371 Date: Aug. 3, 2022

(30) Foreign Application Priority Data
Feb. 11, 2020 (SE) 2050143-3

(51) Int. Cl. A61B 5/15; G03F 7/00

(52) U.S. Cl. CPC A61B 5/150022; A61B 5/150419; A61B 5/150984; G03F 7/0035

(57) ABSTRACT

Microneedle (100, 200, 300, 720) comprising an elongated body (110, 210, 310) extending along a longitudinal axis from a top end to a bottom end on a substrate (300, 710), where the elongated body comprises an upper portion (120, 220, 320) and a lower portion (130, 230, 330). The lower portion (130, 230, 330) comprises an internal capillary bore hole (260, 730) extending into the substrate (300, 710). The upper portion (120, 220, 320) of the elongated body (110, 210, 310) has a semi-enclosed internal void space (140, 240) formed by at least three body sides whereof two body sides join at a sharp edge and a third body side is provided with an opening slit (150, 250, 350) extending from the lower portion (130, 230, 330) of the elongated body (110, 210, 310) to the upper end of the third body side. The top end of the elongated body (110, 210, 310) is configured as a bevel to create a sharp tip at the top of said edge, said bevel extending to the third body side. The semi-enclosed internal void space (140, 240) of the upper portion opening to the internal capillary bore hole of the bottom end of the elongated body (110, 210, 310), and the bottom end of the elongated body is connected to the substrate (300, 710).

### TECHNICAL FIELD

The present invention relates in general to a microneedle and a fluid channel system, and a chip comprising at least one microneedle and a fluid channel system, for sampling of bodily fluids. The present invention relates in particular to at least one microneedle provided on a substrate having an elongated body that comprises an internal capillary bore hole, wherein a top end of the elongated body is configured as a bevel to create a sharp tip at the top of said edge, and wherein the bevel is provided with an opening slit extending from the capillary bore hole to a body side and the bevel. The present invention also relates to a fluid channel system for transporting fluid from a microneedle capillary bore hole to a fluid collection area via a fluid channel, comprising a plurality of channels, a fluid collection area, a first droplet formation structure that is arranged to prevent fluid moving from a first channel into any other first channel, and a second droplet formation structure. The present invention also relates to a chip for collecting fluid via at least one microneedle and a fluid channel system. The present invention also relates to a method of fabricating a chip comprising a at least one microneedle and a fluid channel using a micro electro mechanical system fabrication process.

### BACKGROUND ART

There are numerous ways to sample bodily fluids, for example by using a standard hypodermic needle. This is cumbersome and improved alternatives exist, such as by using hollow microneedles. Although many application fields exist for microneedles, the vast majority of published microneedles concern drug delivery in various forms.

For example the concept of an array of miniaturized needles for drug delivery purposes dates back to the 70's U.S. Pat. No. 3,964,482. One of the earliest reported microneedles in the scientific literature was an out-of-plane silicon needle array featuring 100, 1.5 mm long, needles on an area of 4.2 mm×4.2 mm in “A silicon-based, three-dimensional neural interface: manufacturing processes for an intercortical electrode array.”, IEEE Trans Biomed Eng. 1991 August; 38(8):758-68, Campbell et al. Eventually bio sensing technology will be to the 21st century what microelectronics was to the second half of the 20th century.

Integrated circuits (IC) have had an enormous impact on our daily life today and making use of the same miniaturization and cost benefits of volume manufacturing bio sensing has the potential to move clinical diagnosis and health monitoring from expensive laboratories to small hand-held consumer devices. Sampling of an analyte to be measured is a prerequisite for bio sensing. Many of the designs described in scientific papers have the purpose of extracting bodily fluids, such as blood or interstitial fluid, ISF. Different bodily fluids demands a variety of solutions, for example successful extraction of blood has been demonstrated with use of the natural “overpressure” in the vascular system, while successful extraction of ISF without under-pressure, through diffusion or other mechanisms are rare or even non-existing. The terms “under-pressure” and “sub-pressure” are used as equivalents in the present disclosure.

Independent of method of sampling, the sample has to be transferred to the sensing device in a controlled manner. In order to further improve usage and prevent mistakes, this can preferably performed in an integrated unit.

Microfluidic systems are suitable for transporting body fluids sampled at a multitude of sampling sites, such as a microneedle, to a desired location. However, most described microfluidic systems are configured to transport fluid through fully filled fluid channels and may not function satisfactory for extremely low flows of bodily fluid.

In order to transfer a fluid sample having a limited volume in a controlled manner from a microneedle to a fluid collection area, a design of a suitable fluid channel system is needed.

### SUMMARY OF THE INVENTION

The aim of the present invention is to set aside the abovementioned drawbacks and shortcomings of the previously known microneedles and fluid channel systems, and to provide an improved solution for sampling of bodily fluids.

In order to improve the extraction of fluids, such as ISF, an under-pressure can be applied. This is however difficult to use in all situations, such as in a combination with collecting readings from an integrated sensor. It has been realized that a fluid path extending through a sensor assembly is needed. In addition, in order to operate with limited sampling volumes and further improve the extraction of fluids there is a need of an improved design of the microneedles and/or fluid channel systems to increase the volume from which the sample is collected and/or further improve how the fluid sample is transferred to a fluid collection area.

An object of the invention is to provide an improved microneedle having a fluid channel that allows easy sampling of bodily fluid, such as interstitial fluid, ISF.

Another object of the invention is to also provide a fluid channel system for transporting fluid from a microneedle to a fluid collection area via a fluid channel that allows easy sampling of bodily fluid.

Another object of the invention is to also provide a chip for collecting fluid via at least one microneedle and a fluid channel system that allows easy sampling of bodily fluid.

Another object of the invention is to also provide a chip for collecting fluid via at least one microneedle and a fluid channel system that allows sampling of extremely low flows of bodily fluid. Another object of the invention is to provide a method of fabricating a chip comprising a at least one microneedle and a fluid channel using a micro electro mechanical system fabrication process.

The object of the invention is met in a microneedle provided on a substrate, a fluid channel system, a chip and/or a method as defined in the appending claims.

In a first aspect, the present invention relates to microneedle provided on a substrate. The microneedle comprises an elongated body extending along a longitudinal axis from a top end to a bottom end on the substrate. The elongated body comprises a upper portion and a lower portion. The lower portion of the elongated body comprises an internal capillary bore hole extending into the substrate. The upper portion of the elongated body has a semi-enclosed internal void space formed by at least three body sides whereof two body sides join at a sharp edge and a third body side is provided with an opening slit extending from the lower portion of the elongated body to the upper end of the third body side.

The top end of the elongated body is configured as a bevel to create a sharp tip at the top of said edge, said bevel extending to the third body side. The semi-enclosed internal void space of the upper portion of the elongated body opening to the internal capillary bore hole of the bottom end of the elongated body. The bottom end of the elongated body is connected to the substrate.

By providing the bevel with an opening, the fluid path is extended to the edge of the microneedle in a perpendicular direction relative the longitudinal direction of the microneedle.

This has the advantage of allowing fluid in contact with the microneedle to more easily reach the internal capillary bore hole, thereby allowing an increased amount of fluid to be transported through the microneedle. This further has the advantage of allowing extremely low flows of bodily fluid to be transported through the microneedle, such as flows along one side or interior corner of the internal capillary bore hole.

In embodiments, the opening slit may be positioned at the centre of a flat body side.

This has the advantage of allowing fluid in tissue at the flat body side to more easily reach the internal capillary bore hole. The microneedle inserted into the skin may displace tissue, whereby the displaced tissue pressing against the flat body side provides fluid.

In embodiments, the opening slit may be centred at a flat body side. For example, centred may be at the absolute centre of the body side, alternatives where the opening slit extends over the centre of the body side are also possible.

In embodiments, the opening slit may be located off-set from the centre of the body side.

In embodiments, the at least one surface extending from the semi-enclosed internal void space to the third body side may be curved.

This has the advantage of allowing the fluid at the curved surface to more easily reach the internal capillary bore hole. This further has the advantage of allowing fluid to be guided to the internal capillary bore hole along predetermined regions of the curved surface.

In embodiments, at least a part of all surfaces extending from the semi-enclosed internal void space to the third body side may be curved.

In embodiments, all surfaces extending from the semi-enclosed internal void space to the third body side may be connected through curved surface sides.

In embodiments, at least one surface extending from the semi-enclosed internal void space to the third body side may be connected through a curved surface.

This has the advantage of allowing the fluid at the curved surfaces to more easily reach the internal capillary bore hole. This further has the advantage of allowing fluid to preferentially reach the internal capillary bore hole via predetermined regions of the curved surfaces.

In embodiments, the elongated body may comprise three body sides.

This has the advantage of allowing the microneedle to have two flat body sides forming a piercing geometry, and the third body side comprises the opening slit and is configured to guide fluid to the internal capillary bore hole.

In embodiments, the elongated body may have three body sides. By this, a cutting edge and a sharper microneedle may be designed.

In embodiments, the elongated body may have a triangular or generally triangular cross section. By this, a cutting edge and a sharper microneedle may be designed.

In embodiments, the tip of the microneedle is formed where two body sides are joined.

In embodiments, the two body sides may be joined at a sharp edge are joined to a third body side through curved surfaces.

This has the advantage of allowing the curved surfaces to guide fluid to the internal capillary bore hole via the open slit at the third body side.

In embodiments, the microneedle may comprise ridges that extends on an outer surface of the microneedle, where the ridges extends in a direction that is perpendicular to the longitudinal axis.

This has the advantage of allowing fluid to via at least one ridge more easily reach the internal capillary bore hole. This further has the advantage of allowing the microneedle penetrating skin to keep tissue away from the groves between the ridges, thereby allowing fluid to gather between the ridges.

In embodiments, the upper portion may have a first cross-sectional area, and the lower portion may have a second cross-sectional area, wherein the second cross-sectional area may be larger than the first cross sectional area.

This has the advantage of allowing the microneedle to penetrate the skin with decreased discomfort compared to a microneedle with the second cross-sectional area along the whole elongated body. This further has the advantage of allowing a larger cross section at the lower portion, thereby allowing improved structural integrity of the microneedle.

In embodiments, the microneedle may comprise a dividing plane dividing the upper portion and the lower portion, wherein at least a part of the dividing plane is not in parallel with the substrate plane.

In embodiments, the microneedle may comprise a dividing plane dividing the upper portion and the lower portion, wherein the dividing plane is not in parallel with the substrate plane.

This has the advantage of allowing an upper portion of the elongated body to comprise a cutting edge and the open slit, wherein the cutting edge and the open slit end at different distances from the substrate plane. This further has the advantage of allowing the geometry of the elongated body at the dividing plane to deform tissue in a desirable way and/or guide fluid to the internal capillary bore hole via the open slit.

In embodiments, the dividing plane between the upper portion and the lower portion may be parallel with the bevel.

This has the advantage of allowing the manufacturing of the bevel and the geometry of the elongated body at the dividing plane to be simplified. This has further the advantage of allowing the bevel penetrating the skin and a subsequent passage of the part of the elongated body relating to dividing plane through the plane of the skin to deform the tissue in a similar fashion, thereby limiting discomfort and damage.

In embodiments, the microneedle may comprise a curved portion connecting the elongated portion with the substrate

This has the advantage of allowing improved stability of the elongated portion.

In embodiments, the bevel extending to the third body side may connect with the third body side with an angled portion.

In embodiments, the bevel extending to the third body side may connect with the third body side with a curved portion.

In embodiments, the bevel extending to the third body side may connect with the third body side with a protruding ledge.

In embodiments, the microneedle may be integrally formed on a first substrate of a sensor assembly comprising the first substrate, a second substrate and a sensor. Such sensor assemblies are disclosed in co-pending patent application SE1950886-0, incorporated herein by reference. The first substrate comprises at least one capillary bore hole defining a fluid path. The fluid path is extending through said first substrate from a top side to a fluid channel on a bottom side of said first substrate. The second substrate is arranged in connection with the first substrate and the fluid channel of the first substrate is in fluid communication with a first metallised via and a second metallised via formed in the second substrate. Thereby extending the fluid path through the first metallised via and the second metallised via. The sensor comprises a first electrode and a second electrode. The first electrode and the second electrode is arranged on the second substrate in fluid communication with the fluid channel of the first substrate. The first electrode is in electric contact with the first metallised via and the second electrode is in electric contact with the second metallised via.

In embodiments, the sensor assembly may comprise at least one microneedle or a plurality of microneedles.

In embodiments, the second substrate may be arranged in connection with the first substrate on a side opposite the plurality of the microneedle, the at least one microneedle or the plurality of microneedles.

In embodiments, the first electrode may be shaped as a spiral and the second electrode may be shaped as a spiral, and wherein the spiral shapes of the first and second electrodes are nested.

In embodiments, the sensor is located on a side of the second substrate directed towards the first substrate.

In embodiments, the sensor may at least partly be located on the side of the second substrate that is directed towards the first substrate.

In embodiments, the sensor may at least partly located in the via.

In embodiments, the via may further comprise a signal path that may be extending through the second substrate and the sensor may be arranged in electrical connection with the signal path.

In embodiments, the via may be hollow, thereby providing fluid communication between two opposite sides of the second substrate.

In embodiments, the sensor may be an electrochemical sensor. Electrochemical sensors are known in the art. One known type of electrochemical glucose sensor is the Clark biosensor. This sensor is based on a thin layer of glucose oxidase (GOx) on an oxygen electrode. The readout is the amount of oxygen consumed by GOx during enzymatic reaction with the substrate glucose. A more detailed description of biosensors, such as the Clark type, can be found in Anthony P. F. Turner: Biosensors: sense and sensibility, Chem. Soc. Rev., Volume 42, Number 8, 21 Apr. 2013, pages 3175-3648. The described sensors may be adapted for use in the present invention. In such embodiments, at least one electrode of the sensor is coated with an enzyme, such as a redox enzyme. Enzymes useful in the present invention are oxidoreductases acting on an electron donor with oxygen as acceptor. Such enzymes are generally classified in the group EC 1.X.X.X in the enzyme nomenclature of the International Union of Biochemistry and Molecular Biology. One preferred enzyme is glucose oxidase (EC 1.1.3.4) and another is glucose dehydrogenase (EC 1.1.1.47).

In embodiments, the sides of the via may further comprise a substance with a specified surface energy. By this, the flow behaviour of the fluid may be controlled by its interaction with the specified surface energy.

In embodiments, the wall surface of the via may further be made hydrophobic, such as by coating with a hydrophobic substance.

In embodiments, the wall surface of the via may further be made hydrophilic, such as by coating with a hydrophilic substance.

In embodiments, a cross-sectional area of the capillary bore hole in the distal end may be larger than the cross-sectional area of the capillary bore hole in the proximal end.

In embodiments, the fluid channel may further comprise a decreasing cross-sectional area in order to further enhance a fluid flow in the fluid channel.

The term cross-sectional area means the area of a cross section of an object. The cross section is the intersection of the object and a plane. In comparing different cross-sectional area it is understood that the intersecting planes are parallel unless otherwise stated.

In embodiments, the first substrate may further comprise a frame located on the same side as the microneedle, the at least one microneedle or the plurality of microneedles, wherein the frame at least partly surrounds an area on the first substrate having the plurality of microneedles.

In embodiments, the frame may have a height in a direction extending from the substrate that is equal or higher than the height of the plurality of microneedles.

In embodiments, the cross-sectional area of the capillary bore hole of the microneedles may gradually decrease from the distal end towards the proximal end along the longitudinal direction. This contributes to an enhanced fluid flow through the capillary bore hole, by means of capillary force acting on the fluid in the capillary bore hole.

In embodiments, the cross-section (crosswise to the longitudinal direction) of the capillary bore hole may further comprise at least one rounded corner. This contributes to the wetting of the capillary bore hole, which has a positive effect on the fluid flow.

In embodiments, the capillary bore hole may have a triangular cross-section. A triangular cross-section has been demonstrated to provide a very good fluid flow in the capillary bore hole. A triangular cross-section within this application encompasses cross sections with substantially triangular shape, i.e. edges with convex or concave shape or straight shape, corners with sharp angles and corners with blunt angles as well as rounded corners.

In embodiments, the capillary bore hole may have a cross-section comprising multiple sharp corners and edges along the internal walls, thereby resulting in a cross-section in the form of for example a multiple pointed star polygon, a ring of flower petals, a MEMS comb drive structure, saw-tooth structures, a hypocycloid shape, an astroid shape, a bicorn shape, or a tricuspoid shape.

In embodiments, the walls of the capillary bore hole may comprise hydrophilic surfaces, which enhances the fluid flow in the capillary bore hole.

In embodiments, the fluid channel may be configured to provide an under-pressure, relative the atmospheric pressure, to the capillary bore hole, whereby fluid flow through the capillary bore hole is enhanced. An under-pressure may for example be created with a syringe or a vacuum pump connected to the fluid channel. An under-pressure may also be created with a suction applying device as described in WO2019/020327, incorporated by reference herein, connected to the fluid channel.

In embodiments, the elongated body of the microneedle may further comprise a lateral hole extending in a radial direction relative the longitudinal direction, wherein the lateral hole is in fluid communication with the capillary bore hole. This has the effect that the risk for clogging in the capillary bore hole is reduced.

In embodiments, the second substrate may be operatively connected to the first substrate by means of anodic/direct bonding, which provides a strong and fluid tight seal without the risk of clogging the fluid channels with adhesive.

In embodiments, a chip comprising one or more microneedles according to the invention can be incorporated into a sensor assembly, such as a sensor assembly described in co-pending application SE1950886-0, incorporated herein by reference. Such a chip or sensor assembly may further be comprised in a measurement device. The measuring device may further comprise a suction device, such as disclosed in WO2019/020327, arranged in connection with the chip or sensor assembly on a side opposite the one or plurality of microneedles and in fluid communication with the capillary bore hole of the microneedle, thereby providing a pressure difference through the fluid channel of the microneedles and chip and/or sensor assembly.

In embodiments, the one or plurality of microneedles integrally formed on the first substrate may comprise an elongated body extending from a distal end with a bevel to a proximal end on the substrate along a longitudinal axis. The elongated body may comprise a capillary bore hole extending in a longitudinal direction thereof and that defines a fluid path. The proximal end may be integrally connected with the substrate and the capillary bore hole may be in fluid communication with a fluid channel of the first substrate. Further, the cross-sectional area of the capillary bore hole in the distal end may be larger than the cross-sectional area of the capillary bore hole in the proximal end.

A bevel is referred to as a beveled surface relative the longitudinal axis of the capillary bore hole.

In embodiments, the one or plurality of microneedles and/or the first substrate may comprise silicon.

In embodiments, the one or plurality of microneedles and/or the first substrate may comprise a majority of silicon.

In embodiments, the one or plurality of microneedles and/or the first substrate may be made of silicon.

In embodiments, at least one side of the semi-enclosed internal void space of the upper portion and at least one side of the internal capillary bore hole of the bottom end of the elongated body may form a continuous surface.

In embodiments, at least two sides of the semi-enclosed internal void space of the upper portion and at least two sides of the internal capillary bore hole of the bottom end of the elongated body may form a continuous surface.

In embodiments, all sides of the semi-enclosed internal void space of the upper portion may form respective continuous surfaces with sides of the internal capillary bore hole of the bottom end of the elongated body.

In embodiments, the capillary bore hole of the bottom end of the elongated body may merge continuously with the internal void space of the upper portion of the elongated body on at least one side.

In embodiments, the capillary bore hole of the bottom end of the elongated body may merge continuously with the internal void space of the upper portion of the elongated body on at least two adjacent sides.

In embodiments, the opening between the internal void space of the upper portion and the internal capillary bore hole of the bottom end may have an area equal or similar to the area of the internal capillary bore hole.

In embodiments, the opening between the internal void space of the upper portion and the internal capillary bore hole of the bottom end may be aligned with the internal capillary bore hole.

The opening between the internal void space of the upper portion and the internal capillary bore hole of the bottom end may for example be the area that is common to both the internal void space of the upper portion and the internal capillary bore hole of the bottom end, where the internal void space of the upper portion meets the internal capillary bore hole of the bottom end, when observed in a direction along the longitudinal axis of the microneedle.

In embodiments, the semi-enclosed internal void space of the upper portion may be a continuation of the internal capillary bore hole of the bottom end.

In embodiments, the internal capillary bore hole may extend essentially straight through the substrate.

In embodiments, the size of the internal capillary bore hole on one side of the substrate may be of the same size of the internal capillary bore hole on the opposite side of the substrate. The size of the internal capillary bore hole on one side of the substrate may also be within 1%, 5%, 10%, 25%, 50%, or 100% of the size of the internal capillary bore hole on the opposite side of the substrate. The size may be a diameter, but may also be a cross sectional area.

In embodiments, the cross sectional area of the internal capillary bore hole may increase linearly while extending through the substrate.

In embodiments, the sides of the internal capillary bore hole may be straight.

In embodiments, the sides of the internal capillary bore hole may be straight while extending through the substrate.

In embodiments, the internal capillary bore hole may form an edge on a boundary with the substrate on a side opposite of the microneedle.

In embodiments, the cross section of the internal capillary bore hole may be triangular, square, drop shaped, star shaped, or circular.

In a second aspect, the present invention relates to a fluid channel system for transporting fluid from a plurality of inlets to a fluid collection area via a fluid channel, the fluid channel system comprising the fluid collection area and at least one sector. Each sector comprises a first section and a second section, wherein in each sector the first section comprises at least two inlet channels each connecting an inlet and the second section. In each sector the second section connects the at least two inlet channels of the first section and the fluid collection area via a connective channel, wherein in each sector the at least two inlet channels merge into the connective channel.

Each sector comprises at least one first droplet formation structure between the at least two inlets channels. The at least one first droplet formation structure is arranged to collect fluid by reducing the fluid interface area to air and enlarge the fluid interface area in contact with the channel wall via channel wall geometry, moving from one inlet channel towards another inlet channel than the one inlet channel.

Each connective channel comprises a second droplet formation structure, wherein the second droplet formation structure is arranged at the end of the connective channel providing an outlet to the fluid collection area and the second droplet formation structure is arranged to collect fluid moving from at least one connective channel.

The fluid channel is defined at least by the at least two inlet channels and the at least one connective channel.

This has the advantage of allowing improved collection of a limited amount of fluid from an inlet. This further has the advantage of allowing extremely low flows or sporadic flows of bodily fluid to be transported through fluid channel system, such as flows along one side or interior corner of a fluid channel.

In embodiments, the at least one first droplet formation structure may be arranged to release collected fluid towards the connective channel.

In embodiments, the at least one first droplet formation structure may be arranged to release collected fluid towards the connective channel via a meeting structure. The meeting structure may for example be a channel wall extruded from a wall of the connective channel towards the droplet formation structure. The channel wall may be with a curvature or without a curvature.

In embodiments, the fluid channel system may further comprise at least two sectors.

In embodiments, the fluid channel system may comprise at least three sectors.

This has the advantage of allowing an increased number of capillary bore holes of microneedles able to connect to the fluid channel system. This further has the advantage of limiting the volume and/or length of the fluid channel for transporting fluid from a microneedle capillary bore hole to a fluid collection area, thereby limiting the amount of fluid retained in the fluid channel system.

In embodiments, each first section may connect at least three inlets to the fluid collection area via the connective channel.

This has the advantage of allowing an increased number of inlets of the fluid channel system.

In embodiments, each sector may comprise a plurality of first sections, wherein each one of the plurality of first sections similar to the first section.

In embodiments, each first droplet formation structure for each two inlet channels may comprise two adjacent curved walls joined at an angle of less than 180°, such as less than 120°, 90°, 45°, 30°, 15° or 10°.

In embodiments, at least one first droplet formation structure may comprise a surface treated region at least part of the wall of the fluid channel between the at least two inlets, wherein the surface treated region is arranged to direct fluid to the fluid collection area.

This has the advantage of allowing partial flow of fluid from an inlet to be guided to the fluid collection area and be guided away from another inlet. This further has the advantage of limiting the amount of fluid retained in the fluid channel system for small flows of fluid, thereby allowing an increased amount of a small amount fluid entering the fluid channel system to reach the fluid collection area.

In embodiments, each second droplet formation structure may comprise two adjacent curved walls joined at an angle of less than 180°, such as less than 120°, 90°, 45°, 30°, 15° or 10°.

In embodiments, at least one second droplet formation structure may comprise a surface treated region at the wall of the second channel arranged to retain fluid flowing towards the fluid collection area.

This has the advantage of allowing fluid flowing towards the fluid collection area to form droplets of fluid at each second droplet formation structure, whereby the fluid droplets may be accessed and collected from the fluid collection area. This further has the advantage of allowing the fluid collection area to be open to the environment and allow easy access to fluid droplets formed any second droplet formation structure, while limiting the amount of undesired interaction between the fluid and the environment, such as evaporation.

In embodiments, at least one second droplet formation structure may comprise a chemical substance arranged to interact with collected fluid and limit undesired reactions, such as citrate interacting with blood. The chemical substance to interact with collected fluid may be comprised in a surface layer at the second droplet formation structure.

In embodiments, at least one inlet channel may comprise a first inlet channel part connecting the inlet and the first droplet formation structure, wherein the first inlet channel part geometry comprise at least two adjacent walls joined at an angle of less than 180°, wherein the inlet channel part longitudinally connects to the inlet.

By this, transfer of fluids from the inlet through the inlet channel towards the droplet formation structures may be improved. The transfer of fluid may be in intermittent and low-volume flows, corner flows, Concus-Finn flows, as well as partly and fully developed flows.

In embodiments, each inlet channel may comprise a first inlet channel part connecting the inlet and the first droplet formation structure, wherein the first inlet channel part geometry comprise at least two adjacent walls joined at an angle of less than 180°, wherein the inlet channel part longitudinally connects to the inlet.

In embodiments, the inlet may for example be a part of a bore hole or an upstream connective channel.

This has the advantage of allowing flow to reach the first droplet formation structure along a predetermined side of the first inlet channel part. The first droplet formation structure may be arranged to allow flow along a predetermined side or interior corner of the first inlet channel part to pass through the region of the first channel comprising the first droplet formation structure, and arranged to limit flow along other sides or interior corners of the first inlet channel part.

In embodiments, the cross-sectional area of the connective channel may decrease from the inlets to the fluid collection area.

In embodiments, the width of the connective channel may decrease from the inlets to the fluid collection area. Decreasing the channel width may be done by photolithographic mask design.

In embodiments, the depth of the connective channel may decrease from the inlets to the fluid collection area. Decreasing the channel depth may be done by photolithographic mask design.

In embodiments, each connective channel may have a tapered channel geometry decreasing in cross-sectional area from the substrate backside inlets to the fluid collection area, both by decreasing the channel width by photolithographic mask design, and, as a result from processing of the decreased channel width, decreasing the channel depth.

This has the advantage of allowing fluid to flow along at least one side or interior corner of the connective channel and allowing a fluid droplet to form at the second droplet formation structure at the end at the fluid collection area. This further has the advantage of allowing a lower amount of fluid to completely fill the end of the second channel at the fluid collection area.

In embodiments, the fluid collection area further may comprise an exit port, wherein the exit port is arranged to allow extraction of fluid collected in the fluid collection area.

This has the advantage of allowing fluid entering the fluid channel system to gather over time at the second droplet formation structures, thereafter fluid collected at the second droplet formation structures may be accessed via the exit port and extracted from the fluid channel system. This further has the advantage of allowing a device arranged to provide active fluid flow, such as a pump providing a sub pressure, to interface with the fluid channel system via the exit port and actively transport fluid collected at the second droplet formation structures from the fluid channel system.

In embodiments, at least a part of a channel may have a hydrophilic interior surface.

The hydrophilic surface may also be referred to as a high-energy surface.

The part having a hydrophilic interior surface may be a part of any one inlet channel, any one connective channel, a plurality of the inlet channels, a plurality of the connective channels, all of the inlet channels, all of the connective channels. The part may further be a part of one sector, a plurality of sectors or all sectors. Similarly, the part may further be a part of one section, a plurality of sections or all sections. The part may be a part of a collection area, a plurality of collections areas or all collection areas.

In embodiments, at least one side of a channel may have a hydrophilic interior surface.

In embodiments, at least one side of a channel through the fluid channel system may have a hydrophilic interior surface.

In embodiments, at least two adjacent walls of the cross-sectional channel walls of a channel may have a hydrophilic surface.

The channel walls having a hydrophilic interior surface may be a part of any one inlet channel, any one connective channel, a plurality of the inlet channels, a plurality of the connective channels, all of the inlet channels, all of the connective channels. The channel walls may further be a part of one sector, a plurality of sectors or all sectors. Similarly, the channel walls may further be a part of one section, a plurality of sections or all sections. The channel walls may be a part of a collection area, a plurality of collections areas or all collection areas.

By having at least two adjacent walls hydrophilic, corner flows may be improved.

The term corner flows means fluid flows along or at the edge or corner formed at the joint of at least two surfaces or walls.

By having a hydrophilic interior surface in a channel, the filling may be improved. Thereby corner flows, intermediate flows and fully developed flows may be improved. In embodiments, the high-energy surfaces may comprise of a high-energy surface material bonded to the backside of the substrate thus covering the fluidic system except above the exit port.

In embodiments, the fluid channel system may further comprise a lid attached to the substrate having the channel system. The lid may be arranged to operate as a cover over the fluidic system.

In embodiments, the lid or cover may cover the backside of the substrate, thus covering the fluidic system, except above the exit port.

In embodiments, the lid or cover may comprise a high-energy surface material. The lid or cover may for example be glass. The lid or cover may for example be attached to the substrate by bonding or gluing.

In embodiments, the lid or cover may comprise a high-energy material covering at least a part of the fluid channel system. The lid or cover may for example comprise a hydrophobic material that may be treated to be hydrophilic on parts of the surface, such as the surface covering the fluid channel system. The hydrophobic material may for example be a plastic and the hydrophilic treatment may for example be a surface treatment or the addition of a hydrophilic material. The lid or cover may for example be attached to the substrate by bonding or gluing.

In a third aspect, the present invention relates to a chip for collecting fluid via at least one microneedle. The chip may comprise at least one microneedle according to any herein described embodiment relating thereto. The at least one microneedle is integrally formed on a first side of a common substrate, wherein each proximal end is integrally formed with the substrate and each capillary bore hole is in fluid communication with a fluid channel system according to any herein described embodiment relating thereto on a second side of the substrate. The chip is arranged to, upon a microneedle brought in contact with fluid, passively transport said fluid though said microneedle and the fluid channel system to a fluid collection area.

This has the advantage of allowing the at least one microneedle in contact with fluid to guide a flow of fluid into the internal capillary bore hole and to a fluid collection area via the fluid channel system. This further has the advantage of allowing extremely low flows of bodily fluid to enter the microneedle and be transported through the fluid channel system, such as flows along one side and/or interior corner of the internal capillary bore hole and/or the fluid channel.

In embodiments, the fluid channel of a chip may comprise directional changes with an angle Θ smaller than 90 degrees. By avoiding sharp bends of the fluid channel, fluid flow is enhanced.

In embodiments, the walls of the fluid channel of a chip may comprise directional changes with an angle Θ smaller than 90 degrees. By avoiding sharp bends of the walls of the fluid channel, fluid flow is enhanced.

In embodiments, the chip may further comprise a base substrate, which comprises a fluid port in fluid communication with the fluid channel, and which opens in the backside of the base substrate. This provides an access point to the sampled fluid which can be connected to a second substrate layer, a sensor element or further fluid channels.

In embodiments, the fluid port may comprise an increasing area in the longitudinal direction of the port towards the back of the base substrate or the second substrate. This improves a fluid tight connection to other fluid channels such as tubes or syringes.

In embodiments, the base substrate may be operatively connected to the substrate by means of anodic/direct bonding, which provides a strong and fluid tight seal without the risk of clogging the fluid channels with adhesive.

In embodiments, the at least one microneedle on the chip may be surrounded by an edge, which may be in level with the distal end of the at least one microneedle. This has the effect that a stretchable material, e.g. soft tissue, of a test subject is tensioned during engagement with the distal end of the at least one microneedle.

In embodiments, the at least one microneedle on the chip may be surrounded by an edge, which may be higher than the distal end of the at least one microneedle. By this the edge may extend further from the first substrate than the distal end of the at least one microneedle. This has the effect that a stretchable material, e.g. soft tissue, of a test subject is tensioned during engagement with the distal end of the at least one microneedle.

In embodiments, the exit port may be located at a central position of the chip.

In a fourth aspect, the present invention relates to a method of fabricating a chip comprising at least one microneedle and a fluid channel using a micro electro mechanical system fabrication process. The method comprising growing a sacrificial oxide layer on a silicon wafer substrate, masking the sacrificial oxide layer with a patterned photoresist, removing the sacrificial oxide layer according to the patterned photoresist, etching the silicon wafer according to the pattern of the removed sacrificial oxide layer using a deep reactive ion etching method, and removing the remaining sacrificial oxide layer by using an etchant.

In one embodiment, the etchant used to remove the sacrificial oxide layer may be a liquid etchant, a plasma etchant, or a combination thereof. A liquid etchant may for example comprise hydrofluoric acid or a buffered oxide etch.

In one embodiment, the chip may comprise silicon.

### BRIEF DESCRIPTION OF THE DRAWINGS

A more thorough understanding of the abovementioned and other features and advantages of the present invention will be evident from the following detailed description of embodiments with reference to the enclosed drawings, on which:

FIG. 1 is a schematic perspective view of a microneedle.

FIG. 2a is a microneedle shown from above according to an embodiment.

FIG. 2b is a microneedle shown from above according to an embodiment.

FIG. 2c is a microneedle shown from above according to an embodiment.

FIG. 2d is a microneedle shown from above according to an embodiment.

FIG. 2e is a microneedle shown from above according to an embodiment.

FIG. 2f is a cross-section of a microneedle according to an embodiment.

FIG. 3a is a schematic perspective view of a microneedle.

FIG. 3b is a schematic perspective view of a microneedle.

FIG. 3c is a schematic perspective view of a microneedle.

FIG. 4 is a schematic view of a fluid channel system shown from above.

FIG. 5 is a schematic view of a fluid channel system shown from above according to an embodiment.

FIG. 6a is a schematic view of a fluid channel system shown from above according to an embodiment.

FIG. 6b is a schematic perspective view of a part of a fluid channel system according to an embodiment.

FIG. 7 is a cross-sectional view of a schematic chip.

FIG. 8 is a schematic illustration of a method of fabricating a chip.

### DETAILED DESCRIPTION

The present invention is based on the insights disclosed. When examined carefully it's clear that there is a need of an improved design of the microneedles and/or fluid channel systems in the prior art for sampling of bodily fluids.

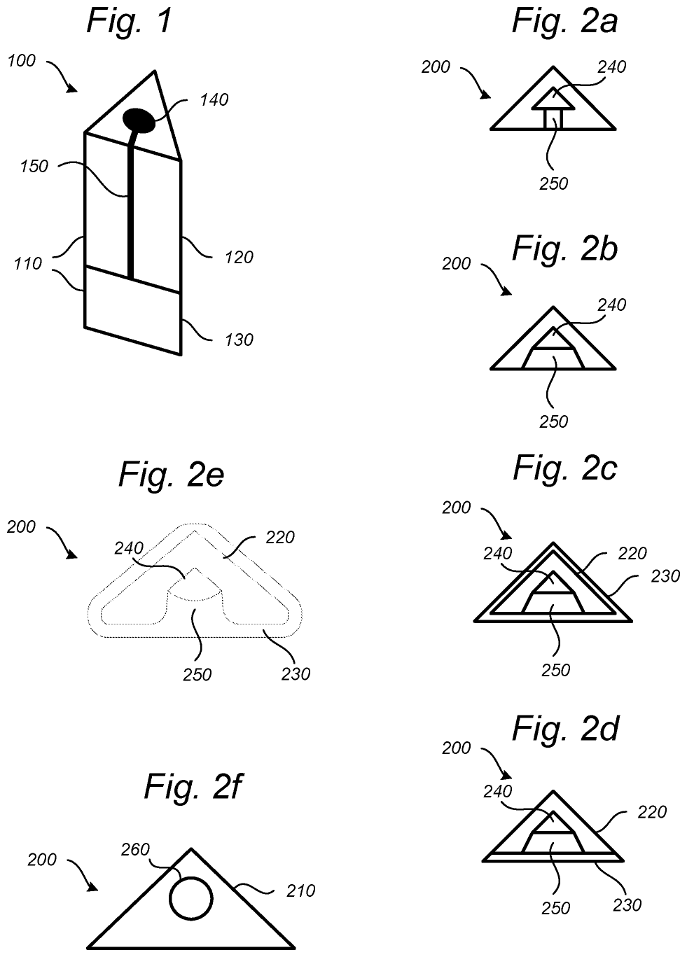

FIG. 1 is a schematic perspective view of a microneedle 100 according to an embodiment of the present invention having an elongated body 110 extending along a longitudinal axis from a top end to a bottom end. The elongated body comprises a upper portion 120 and a lower portion 130. The upper portion 120 of the elongated body 110 has a semi-enclosed internal void space 140 formed by three body sides whereof two body sides join at a sharp edge and a third body side is provided with an opening slit 150 extending from the lower portion 130 of the elongated body 110 to the upper end of the third body side. The top end of the elongated body 110 is configured as a bevel to create a sharp tip at the top of the edge, the bevel extends to the third body side. The semi-enclosed internal void space 140 of the upper portion 120 of the elongated body 110 is opening to an internal capillary bore hole of the bottom end 130 of the elongated body 110. The internal capillary bore hole of the lower portion 130 of the elongated body 110 extends through the microneedle 100 and may also extend into a substrate to which the microneedle 100 may be provided on.

FIG. 2a is a top-view along the longitudinal axis of a microneedle 200 according to an embodiment. The microneedle 200 has a semi-enclosed internal void space 240 formed by three body sides whereof two body sides join at a sharp edge and a third body side is provided with an opening slit 250. The semi-enclosed internal void space 240 of the upper portion of the elongated body is here illustrated as having a triangular cross-section and the opening slit 250 is positioned at the centre of a flat body side. The opening slit 250 shown has a width that is smaller than the width of the internal void space 240.

FIG. 2b is a top-view along the longitudinal axis of a microneedle 200 according to an embodiment. The microneedle 200 has a semi-enclosed internal void space 240 formed by three body sides whereof two body sides join at a sharp edge and a third body side is provided with an opening slit 250. The semi-enclosed internal void space 240 of the upper portion of the elongated body is here illustrated as having a triangular cross-section and the opening slit 250. The opening slit 250 shown has a width that is larger than the width of the internal void space 240.

FIG. 2c is a top-view along the longitudinal axis of a microneedle 200 according to an embodiment. The microneedle 200 has a semi-enclosed internal void space 240 formed by three body sides whereof two body sides join at a sharp edge and a third body side is provided with an opening slit 250. The semi-enclosed internal void space 240 of the upper portion of the elongated body is here illustrated as having a triangular cross-section and the opening slit 250. In the present figure, the lower portion 230 can be seen as the upper portion 220 is having a first cross-sectional area, and the lower portion 230 is having a second cross-sectional area, wherein the second cross-sectional area is larger than the first cross sectional area. By this, the lower portion 230 may extend outside the upper portion 220. In the present figure, the lower portion 230 extend outside the upper portion 220 in an even manner.

FIG. 2d is a top-view along the longitudinal axis of a microneedle 200 according to an embodiment. The microneedle 200 has a semi-enclosed internal void space 240 formed by three body sides whereof two body sides join at a sharp edge and a third body side is provided with an opening slit 250. The semi-enclosed internal void space 240 of the upper portion of the elongated body is here illustrated as having a triangular cross-section and the opening slit 250. In the present figure, the lower portion 230 can be seen as the upper portion 220 is having a first cross-sectional area, and the lower portion 230 is having a second cross-sectional area, wherein the second cross-sectional area is larger than the first cross sectional area. By this, the lower portion 230 may extend outside the upper portion 220. In the present figure, the lower portion 230 extend outside the upper portion 220 on the third body side.

FIG. 2e is a top-view along the longitudinal axis of a microneedle 200 according to an embodiment. The microneedle 200 has a semi-enclosed internal void space 240 formed by three body sides whereof two body sides join at a sharp edge, a third body side is provided with an opening slit 250, and two body sides are joined to the third body side through curved surfaces. The semi-enclosed internal void space 240 of the upper portion of the elongated body is here illustrated as having a triangular cross-section and the opening slit 250.

In the present figure, the surfaces extending from the semi-enclosed internal void space 240 to the third body side are curved. In other examples, only a part of all surfaces, or a part of at least one surface, extending from the semi-enclosed internal void space 240 to the third body side are curved.

In the present figure, the lower portion 230 can be seen as the upper portion 220 is having a first cross-sectional area, and the lower portion 230 is having a second cross-sectional area, wherein the second cross-sectional area is larger than the first cross sectional area. By this, the lower portion 230 may extend outside the upper portion 220. In the present figure, the lower portion 230 extend outside the upper portion 220 in an even manner. The sides of the lower portion 230 are here joined through curved surfaces. In other examples, at least one or a plurality of the joints may be made through curved surfaces, while other may be joint at sharp edges.

Other shapes of the internal void space than the illustrated ones having a triangular cross-section are possible, such as for example the capillary bore hole may have a cross-section comprising multiple sharp corners and edges along the internal walls, thereby resulting in a cross-section in the form of for example a multiple pointed star polygon, a ring of flower petals, a MEMS comb drive structure, saw-tooth structures, a hypocycloid shape, an astroid shape, a bicorn shape, or a tricuspoid shape.

FIG. 2f is a cross-section of a microneedle 200 shown along the longitudinal axis of a microneedle 200 according to an embodiment. The illustrated microneedle 200 has a capillary bore hole 260 with a circular cross-sectional shape. The semi-enclosed internal void space may also have a circular shape.

The internal edges may also be edges with convex or concave shapes or straight shapes, corners with sharp angles and corners with blunt angles as well as rounded corners are also possible.

The walls of the internal void space and/or capillary bore hole may also comprise hydrophilic surfaces to enhance the fluid flow in the space or hole.

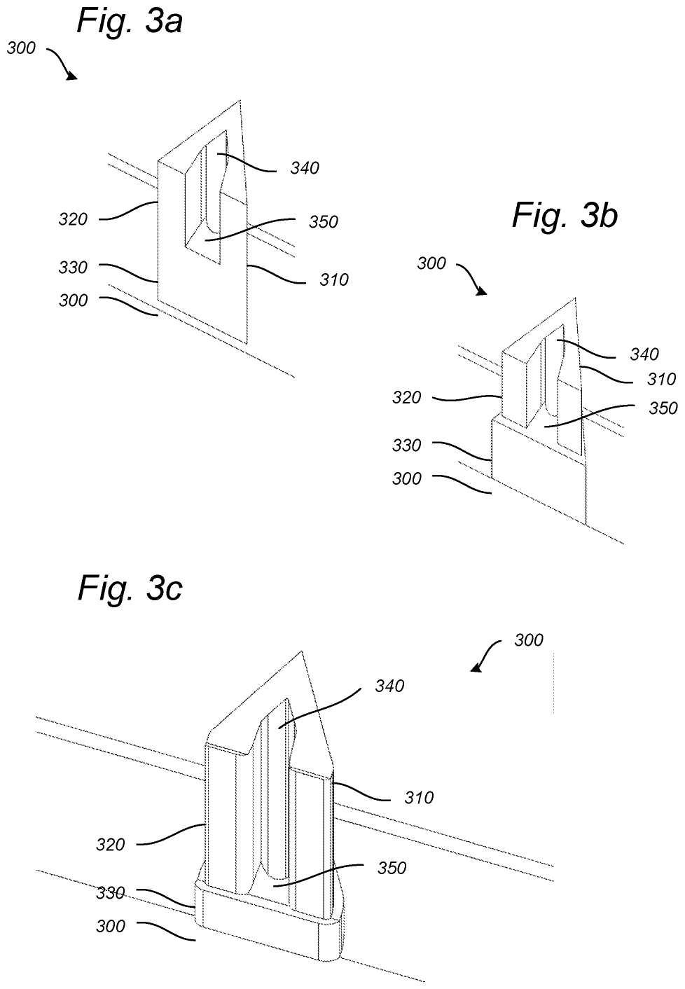

FIG. 3a is a schematic perspective view of a microneedle 300 provided on a substrate according to an embodiment of the present invention having an elongated body 310 extending along a longitudinal axis from a top end to a bottom end. The elongated body comprises a upper portion 320 and a lower portion 330. The upper portion 320 of the elongated body 310 has a semi-enclosed internal void space 340 formed by three body sides whereof two body sides join at a sharp edge and a third body side is provided with an opening slit 350 extending from the lower portion 330 of the elongated body 310 to the upper end of the third body side. The top end of the elongated body 310 is configured as a bevel to create a sharp tip at the top of the edge, the bevel extends to the third body side. The semi-enclosed internal void space 340 of the upper portion 320 of the elongated body 310 is opening to an internal capillary bore hole of the bottom end 330 of the elongated body 310. The internal capillary bore hole of the lower portion 330 of the elongated body 310 extends through the microneedle 300 and may also extend into a substrate to which the microneedle 300 is provided on.

FIG. 3b is a schematic perspective view of a microneedle 300 provided on a substrate according to an embodiment of the present invention having an elongated body 310 extending along a longitudinal axis from a top end to a bottom end. The elongated body comprises a upper portion 320 and a lower portion 330. The upper portion 320 of the elongated body 310 has a semi-enclosed internal void space 340 formed by three body sides whereof two body sides join at a sharp edge and a third body side is provided with an opening slit 350 extending from the lower portion 330 of the elongated body 310 to the upper end of the third body side. The top end of the elongated body 310 is configured as a bevel to create a sharp tip at the top of the edge, the bevel extends to the third body side. The semi-enclosed internal void space 340 of the upper portion 320 of the elongated body 310 is opening to an internal capillary bore hole of the bottom end 330 of the elongated body 310. The internal capillary bore hole of the lower portion 330 of the elongated body 310 extends through the microneedle 300 and may also extend into a substrate to which the microneedle 300 is provided on. In the present figure, the lower portion 330 can be seen as the upper portion 320 is having a first cross-sectional area, and the lower portion 330 is having a second cross-sectional area, wherein the second cross-sectional area is larger than the first cross sectional area. By this, the lower portion 330 may extend outside the upper portion 320. In the present figure, the lower portion 330 extend outside the upper portion 320 on the third body side.

FIG. 3c is a schematic perspective view of a microneedle 300 provided on a substrate according to an embodiment of the present invention having an elongated body 310 extending along a longitudinal axis from a top end to a bottom end. The elongated body comprises a upper portion 320 and a lower portion 330. The upper portion 320 of the elongated body 310 has a semi-enclosed internal void space 340 formed by three body sides whereof two body sides join at a sharp edge and a third body side is provided with an opening slit 350 extending from the lower portion 330 of the elongated body 310 to the upper end of the third body side. The two body sides are further joined to the third body side through curved surfaces, and the surfaces extending from the semi-enclosed internal void space to the third body side are curved. The top end of the elongated body 310 is configured as a bevel to create a sharp tip at the top of the edge, the bevel extends to the third body side. The semi-enclosed internal void space 340 of the upper portion 320 of the elongated body 310 is opening to an internal capillary bore hole of the bottom end 330 of the elongated body 310. The internal capillary bore hole of the lower portion 330 of the elongated body 310 extends through the microneedle 300 and may also extend into a substrate to which the microneedle 300 is be provided on.

In the present figure, the lower portion 330 can be seen as the upper portion 220 is having a first cross-sectional area, and the lower portion 330 is having a second cross-sectional area, wherein the second cross-sectional area is larger than the first cross sectional area. By this, the lower portion 330 may extend outside the upper portion 320. In the present figure, the lower portion 330 extend outside the upper portion 320 on the third body side. The upper portion 320 and the lower portion 330 are divided by a dividing plane. In the figure at least a part of the dividing plane is not in parallel with the substrate plane. In other examples, the dividing plane may also be essentially in parallel with the substrate, the dividing plane may also be parallel with the bevel of the microneedle.

In the present figure, the bevel extending to the third body side connects with the third body side with an angled portion. The angled portion may for example be approximately be in parallel with the substrate. The bevel extending to the third body side may also connect with the third body side with a curved portion.

In additional examples, the elongated body can be connected with the substrate with a curved portion.

In additional examples, the microneedle can also comprise ridges that extends on an outer surface of the microneedle. The ridges may extend in a direction that is perpendicular to the longitudinal axis of the microneedle. In additional embodiments, the ridges may extend in a direction that is in parallel with the longitudinal axis of the microneedle. In additional embodiments, the ridges may be arranged as a spiral extending with the longitudinal axis of the microneedle.

The opening slit may for an example be located at the centre of a body side, such as a flat body side. The opening slit may also be located at an off-set from the centre of a body side.

At least one surface, or at least a part of all surfaces, extending from the semi-enclosed internal void space to the third body side may be curved. In some examples, at least one or even all surfaces extending from the semi-enclosed internal void space to the third body side may be connected through curved surface sides.

The elongated body may comprise three body sides, as illustrated. The elongated body may also be realised with more sides, for example four sides or five sides. Additional examples with even more sides are possible, these more sides may be evenly spaces or asymmetrically spaced. For example, an elongated body having an even number of sides where every other side is longer than the others are possible.

Two body sides of the elongated body may be joined at a sharp edge, while both body sides joint at the sharp edge is joined to a third body side through curved surfaces. The body sides joint at the sharp edge may also be joined to a third body side and a fourth body side, respectively, through curved surfaces.

The microneedle may comprise ridges that extends on an outer surface of the elongated body, where the ridges extends in a direction that is perpendicular to the longitudinal axis.

In illustrations comprising a plurality of sectors, the references may be located at corresponding places in varying sectors.

In additional examples, the one or plurality of microneedles and/or the first substrate may comprise silicon. The one or plurality of microneedles and/or the first substrate may also comprise a majority of silicon, or be made of silicon.

In additional examples, at least one or two sides of the semi-enclosed internal void space of the upper portion and at least one side of the internal capillary bore hole of the bottom end of the elongated body may form a continuous surface. In other examples, all sides of the semi-enclosed internal void space of the upper portion may form respective continuous surfaces with sides of the internal capillary bore hole of the bottom end of the elongated body.

In additional examples, the capillary bore hole of the bottom end of the elongated body may merge continuously with the internal void space of the upper portion of the elongated body on at least one side. The capillary bore hole of the bottom end of the elongated body may also merge continuously with the internal void space of the upper portion of the elongated body on at least two adjacent sides.

In additional examples, the opening between the internal void space of the upper portion and the internal capillary bore hole of the bottom end may have an area equal or similar to the area of the internal capillary bore hole. The opening between the internal void space of the upper portion and the internal capillary bore hole of the bottom end may also be aligned with the internal capillary bore hole.

In additional examples, the semi-enclosed internal void space of the upper portion may be a continuation of the internal capillary bore hole of the bottom end. The internal capillary bore hole may also extend essentially straight through the substrate. The size of the internal capillary bore hole on one side of the substrate may be of the same size of the internal capillary bore hole on the opposite side of the substrate. The size of the internal capillary bore hole on one side of the substrate may also be within 1%, 5%, 10%, 25%, 50%, or 100% of the size of the internal capillary bore hole on the opposite side of the substrate. The size may be a diameter, but may also be a cross sectional area.

In additional examples, the cross sectional area of the internal capillary bore hole may increase linearly while extending through the substrate. The sides of the internal capillary bore hole may also be straight or straight while extending through the substrate.

In additional examples, the internal capillary bore hole may form an edge on a boundary with the substrate on a side opposite of the microneedle.

In additional examples, the cross section of the internal capillary bore hole may be triangular, square, drop shaped, star shaped, or circular.

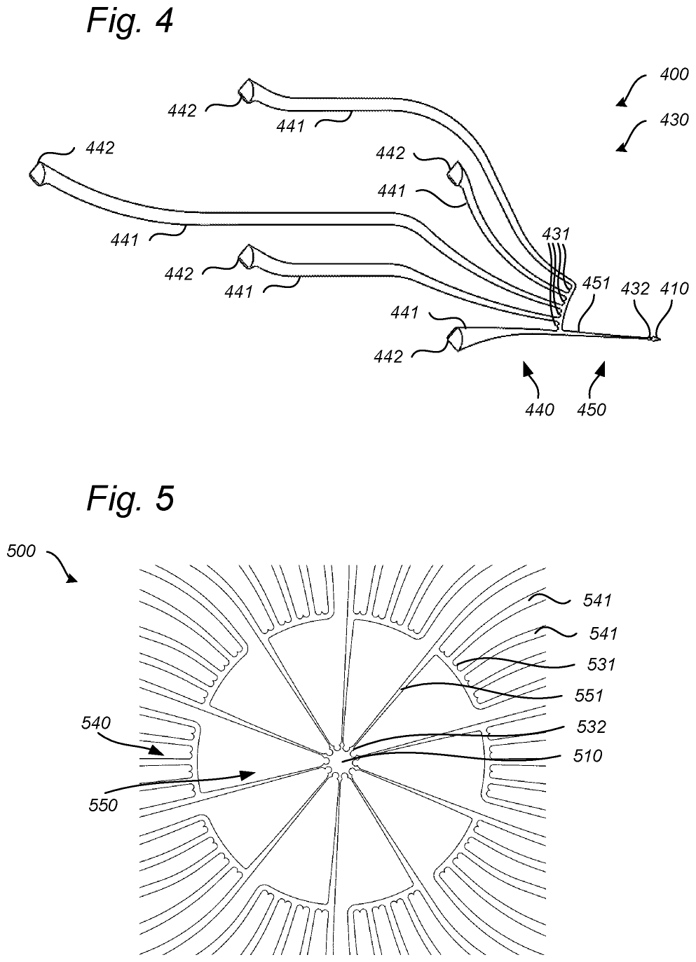

FIG. 4 is a schematic view of a fluid channel system 400 shown from above according to an embodiment. The fluid channel system 400 is arranged for transporting fluid from a plurality of inlets 442 to a fluid collection area 410 via a fluid channel. The fluid channel system 400 illustrated comprise one sector 430, where the sector 430 comprises a first section 440 and a second section 450.

In the sector 430 the first section 440 comprises five inlet channels 441, each inlet channel 441 connecting an inlet 442 and the second section 450. In the sector 430 the second section 450 connects the five inlet channels 441 of the first section 440 and the fluid collection area 410 via a connective channel 451. In the sector 430 the five inlet channels 441 merge into the connective channel 451.

The sector 430 comprises four first droplet formation structures 431 between each of the five inlets channels 441. The four first droplet formation structures 431 are arranged to collect fluid by reducing the fluid interface area to air and enlarge the fluid interface area to the channel wall via channel wall geometry, moving from one inlet channel towards another inlet channel than the one inlet channel.

The connective channel 451 comprises a second droplet formation structure 432. The second droplet formation structure 432 is arranged at the end of the connective channel 451 providing an outlet to the fluid collection area 410 and the second droplet formation structure 432 is arranged to collect fluid moving from the connective channel 451.

FIG. 5 is a schematic view of a fluid channel system 500 shown from above according to an embodiment. The fluid channel system 500 is arranged for transporting fluid from a plurality of inlets to a fluid collection area 510 via a fluid channel. The fluid channel system 500 comprise at least one sector, where each sector comprises a first section 540 and a second section 550.

In each sector the first section 540 comprises at least two inlet channels 541, each inlet channel 541 connecting an inlet and the second section 550. In each sector the second section 550 connects the at least two inlet channels 541 of the first section 540 and the fluid collection area 510 via a connective channel 551. In each sector the at least two inlet channels 541 merge into the connective channel 551.

Each sector comprises at least one first droplet formation structure 531 between the at least two inlets channels 541. The at least one first droplet formation structure 531 is arranged to collect fluid by reducing the fluid interface area to air and enlarge the fluid interface area to the channel wall via channel wall geometry, moving from one inlet channel towards another inlet channel than the one inlet channel.

Each connective channel 551 comprises a second droplet formation structure 532. The second droplet formation structure 532 is arranged at the end of the connective channel 551 providing an outlet to the fluid collection area 510 and the second droplet formation structure 532 is arranged to collect fluid moving from at least one connective channel 551.

The fluid channel has at least two inlet channels 541 and at least one connective channel 551. In the present figure, there are ten sectors. Each sector is provided with five inlet channels 541, four first droplet formation structures 531 and one connective channel 551.

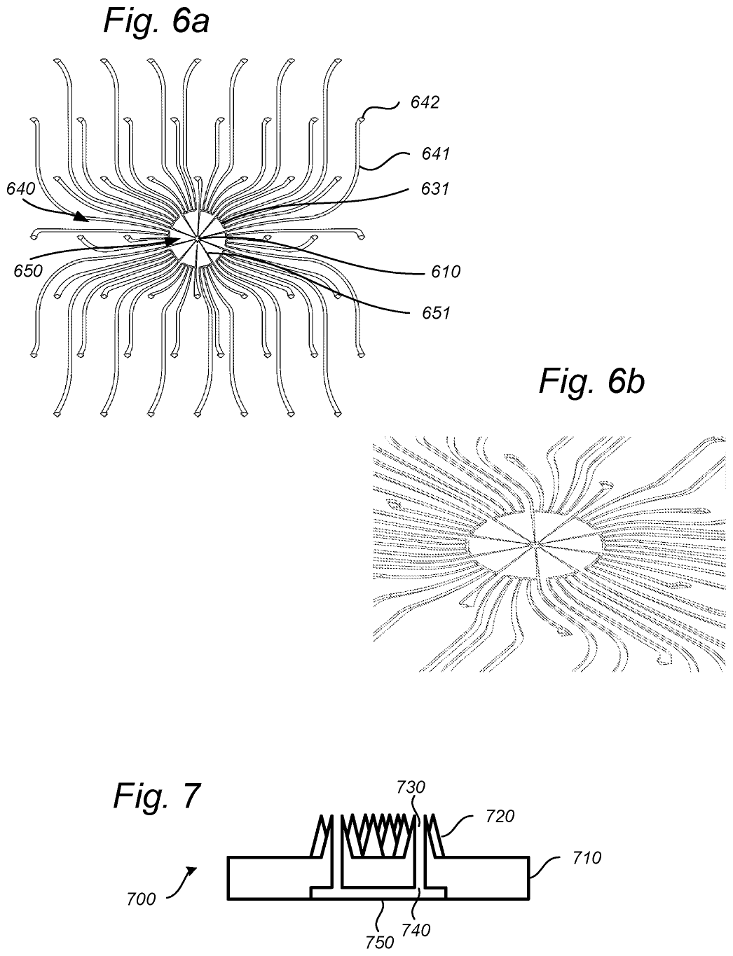

FIG. 6a is a schematic view of a fluid channel system 600 shown from above according to an embodiment. The fluid channel system 600 is arranged for transporting fluid from a plurality of inlets 642 to a fluid collection area 610 via a fluid channel. The fluid channel system 600 comprise at least one sector, where each sector comprises a first section 640 and a second section 650.

In each sector the first section 640 comprises at least two inlet channels 641, each inlet channel 641 connecting an inlet 642 and the second section 650. In each sector the second section 650 connects the at least two inlet channels 641 of the first section 640 and the fluid collection area 610 via a connective channel 651. In each sector the at least two inlet channels 641 merge into the connective channel 651.

Each sector comprises at least one first droplet formation structure 631 between the at least two inlets channels 641. The at least one first droplet formation structure 631 is arranged to collect fluid by reducing the fluid interface area to air and enlarge the fluid interface area to the channel wall via channel wall geometry, moving from one inlet channel towards another inlet channel than the one inlet channel.

Each connective channel 651 comprises a second droplet formation structure. The second droplet formation structure is arranged at the end of the connective channel 651 providing an outlet to the fluid collection area 610 and the second droplet formation structure is arranged to collect fluid moving from at least one connective channel 651.

The fluid channel has at least two inlet channels 641 and at least one connective channel 651. In the present figure, there are ten sectors. Each sector is provided with five inlet channels 641, four first droplet formation structures 631 and one connective channel 651.

FIG. 6b is a part of a schematic view of a fluid channel system 600 shown in perspective according to an embodiment that is similar to the one presents in FIG. 6a.

The channel system may further have at least one first droplet formation structure that can be arranged to release collected fluid towards the connective channel.

Further, the channel system may be arranged so that the at least one first droplet formation structure is arranged to release collected fluid towards the connective channel via a meeting structure. The meeting structure may for example be a channel wall extruded from a wall of the connective channel towards the droplet formation structure. The channel wall may be with a curvature or without a curvature.

The channel system may comprise one, two, at least two, at least three, a plurality, or any suitable number of sectors, such as five, six, seven, eight, nine or ten. Designs having even more sectors are also possible.

The first section may also connect three or more inlets to the fluid collection area via the connective channel. Each sector may in turn comprise a plurality of first sections, wherein each one of the plurality of first sections similar to the first section. The first droplet formation structures for each two inlet channels may also be arranged to comprise two adjacent curved walls joined at an angle of less than 180°, such as less than 120°, 90°, 45°, 30°, 15° or 10°.

The first droplet formation structure may also comprise a surface treated region on at least part of the wall of the fluid channel between the at least two inlets, wherein the surface treated region is arranged to direct fluid to the fluid collection area.

The second droplet formation structure may further comprise two adjacent curved walls joined at an angle of less than 180°, such as less than 120°, 90°, 45°, 30°, 15° or 10°. The second droplet formation structure may also comprise a surface treated region at the wall of the second channel arranged to retain fluid flowing towards the fluid collection area. The second droplet formation structure may further comprise a chemical substance arranged to interact with collected fluid and limit undesired reactions, such as citrate interacting with blood. The chemical substance to interact with collected fluid may be comprised in a surface layer at the second droplet formation structure.

The inlet channel may further comprise a first inlet channel part connecting the inlet and the first droplet formation structure, wherein the first inlet channel part geometry comprise at least two adjacent walls joined at an angle of less than 180°, wherein the inlet channel part longitudinally connects to the inlet. The inlet channel may also comprise a first inlet channel part connecting the inlet and the first droplet formation structure, wherein the first inlet channel part geometry comprise at least two adjacent walls joined at an angle of less than 180°, wherein the inlet channel part longitudinally connects to the inlet. The inlet may for example be a part of a bore hole or an upstream connective channel.

The cross-sectional area of the connective channel may decrease from the inlets to the fluid collection area. To decrease the cross-sectional area of connective channel, the depth of the connective channel may decrease from the inlets to the fluid collection area, and/or the width of the connective channel may decrease from the inlets to the fluid collection area. The connective channel may also have a tapered channel geometry decreasing in cross-sectional area from the substrate backside inlets to the fluid collection area.

The fluid collection area further may be arranged to comprise an exit port, wherein the exit port is arranged to allow extraction of fluid collected in the fluid collection area.

At least a part of a channel or channel wall in the channel system may have a hydrophilic interior surface. The part of the channel or channel wall having a hydrophilic interior surface may be a part of any one inlet channel, any one connective channel, a plurality of the inlet channels, a plurality of the connective channels, all of the inlet channels, all of the connective channels. The part may further be a part of one sector, a plurality of sectors or all sectors. Similarly, the part may further be a part of one section, a plurality of sections or all sections. The part may be a part of a collection area, a plurality of collections areas or all collection areas. For example, at least one side of a channel may have a hydrophilic interior surface, at least one side of a channel through the fluid channel system may have a hydrophilic interior surface, and/or at least two adjacent walls of the cross-sectional channel walls of a channel may have a hydrophilic surface.

The fluid channel system may further comprise a lid attached to the substrate having the channel system. The lid may be arranged to operate as a cover over the fluidic system. The lid or cover may cover the backside of the substrate, thus covering the fluidic system, except above the exit port. The lid or cover may also comprise a high-energy surface material. The lid or cover may for example be glass. The lid or cover may for example be attached to the substrate by bonding or gluing. The lid or cover may also comprise a high-energy material covering at least a part of the fluid channel system. The lid or cover may for example comprise a hydrophobic material that may be treated to be hydrophilic on parts of the surface, such as the surface covering the fluid channel system. The hydrophobic material may for example be a plastic and the hydrophilic treatment may for example be a surface treatment or the addition of a hydrophilic material.

FIG. 7 is a cross-sectional view of a schematic chip 700 for collecting fluid via at least one microneedle. The present figure is illustrated with a plurality of microneedles.

The chip 700 has microneedles according to any herein disclosed embodiment integrally formed on a first side of a common substrate 710. Each proximal end of the microneedles 720 is integrally formed with the substrate 710 and each capillary bore hole 730 is in fluid communication with an inlet 740 of a fluid channel system 750 according to any herein disclosed embodiment on a second side of the substrate 710.

The chip 700 is arranged to, upon a microneedle 720 brought in contact with fluid, passively transport said fluid though said microneedle 720 and the fluid channel system 750 to a fluid collection area.

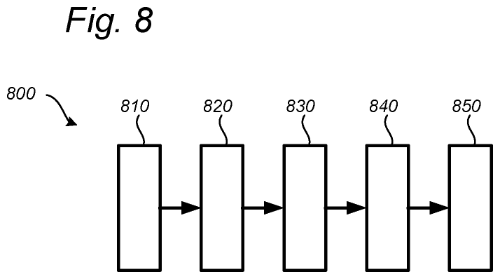

FIG. 8 is a schematic illustration of a method of fabricating a chip comprising at least one microneedle according to any herein disclosed embodiment and a fluid channel according to any herein disclosed embodiment using a micro electro mechanical system fabrication process 800.

The fabrication process illustrated comprises the steps of growing 810 a sacrificial oxide layer on a silicon wafer substrate, masking 820 the sacrificial oxide layer with a patterned photoresist, removing 830 the sacrificial oxide layer according to the patterned photoresist, etching 840 the silicon wafer according to the pattern of the removed sacrificial oxide layer using a deep reactive ion etching method, and removing 850 the remaining sacrificial oxide layer by using an etchant.

The etchant used to remove the sacrificial oxide layer may for example be a liquid etchant, a plasma etchant, or a combination thereof. A liquid etchant may for example comprise hydrofluoric acid or a buffered oxide etch.

Thereby a microneedle, a channel system, a chip, and a fabrication process allowing fluid in contact with the microneedle to more easily reach the internal capillary bore hole, thereby allowing an increased amount of fluid to be transported through the microneedle and a channel system for transporting the fluid, an improved solution for sampling of bodily fluids may be provided.

While specific embodiments have been described, the skilled person will understand that various modifications and alterations are conceivable within the scope as defined in the appended claims.

1. A microneedle provided on a substrate, comprising:
an elongated body extending along a longitudinal axis from a top end to a bottom end on the substrate, wherein:
the elongated body comprises an upper portion and a lower portion;
the lower portion of the elongated body comprises an internal capillary bore hole extending into the substrate;
the upper portion of the elongated body has a semi-enclosed internal void space formed by at least three body sides whereof two body sides join at a sharp edge and a third body side is provided with an opening slit extending from the lower portion of the elongated body to the upper end of the third body side;
the top end of the elongated body is configured as a bevel to create a sharp tip at the top of said edge, said bevel extending to the third body side;
the semi-enclosed internal void space of the upper portion of the elongated body opening to the internal capillary bore hole of the bottom end of the elongated body; and
the bottom end of the elongated body is connected to the substrate.

2. A microneedle according to claim 1, wherein the opening slit is positioned at the centre of a flat body side.

3. A microneedle according to claim 1, wherein at least one surface extending from the semi-enclosed internal void space to the third body side is curved.

4. A microneedle according to claim 3, wherein at least a part of all surfaces extending from the semi-enclosed internal void space to the third body side are curved.

5. A microneedle according to claim 1, wherein the elongated body comprises three body sides.

6. A microneedle according to claim 5, wherein the two body sides joined at a sharp edge are joined to a third body side through curved surfaces.

7. A microneedle according to claim 1, further comprising ridges that extends on an outer surface of the microneedle, where the ridges extends in a direction that is perpendicular to the longitudinal axis.

8. A microneedle according to claim 1, wherein the upper portion is having a first cross-sectional area, and the lower portion is having a second cross-sectional area, wherein the second cross-sectional area is larger than the first cross sectional area.

9. A microneedle according to claim 1, further comprising a dividing plane dividing the upper portion and the lower portion, wherein at least a part of the dividing plane is not in parallel with the substrate plane.

10. A microneedle according to claim 9, wherein the dividing plane between the upper portion and the lower portion is parallel with the bevel.

11. A microneedle according to claim 1, further comprising a curved portion connecting the elongated portion with the substrate.

12. A fluid channel system for transporting fluid from a plurality of inlets to a fluid collection area via a fluid channel, the fluid channel system comprising the fluid collection area and at least one sector, wherein each sector comprises a first section and a second section, wherein in each sector the first section comprises at least two inlet channels each connecting an inlet and the second section, wherein in each sector the second section connects the at least two inlet channels of the first section and the fluid collection area via a connective channel, wherein in each sector the at least two inlet channels merge into the connective channel; wherein
each sector comprises at least one first droplet formation structure between the at least two inlets channels, wherein the at least one first droplet formation structure is arranged to collect fluid by reducing the fluid interface area to air and enlarge the fluid interface area to the channel wall via channel wall geometry, moving from one inlet channel towards another inlet channel than the one inlet channel;
each connective channel comprises a second droplet formation structure, wherein the second droplet formation structure is arranged at the end of the connective channel providing an outlet to the fluid collection area and the second droplet formation structure is arranged to collect fluid moving from at least one connective channel; and
wherein the fluid channel is defined at least by the at least two inlet channels and the at least one connective channel.

13. A fluid channel system according to claim 12, wherein the at least one first droplet formation structure is arranged to release collected fluid towards the connective channel.

14. A fluid channel system according to claim 12, comprising at least two sectors.

15. A fluid channel system according to claim 12, wherein each first section connects at least three inlets to the fluid collection area via the connective channel.

16. A fluid channel system according to claim 12, wherein each first droplet formation structure between each two inlet channels comprises two adjacent curved walls joined at an angle of less than 180°, such as less than 120°, 90°, 45°, 30°, 15° or 10°.

17. A fluid channel system according to claim 12, wherein each second droplet formation structure comprises two adjacent curved walls joined at an angle of less than 180°, such as less than 120°, 90°, 45°, 30°, 15° or 10°.

18. A fluid channel system according to claim 12, wherein each inlet channel comprises a first inlet channel part connecting the inlet and the first droplet formation structure, wherein the first inlet channel part geometry comprise at least two adjacent walls joined at an angle of less than 180°, wherein the inlet channel part longitudinally connects to the inlet.

19. A fluid channel system according to claim 12, wherein the cross-sectional area of the connective channel decreases from the inlets to the fluid collection area.

20. A fluid channel system according to claim 12, wherein the fluid collection area comprises an exit port, wherein the exit port is arranged to allow extraction of fluid collected in the fluid collection area.

21. A fluid channel system according to claim 12, wherein at least a part of a channel has a hydrophilic interior surface.

22. A chip for collecting fluid via at least one microneedle, comprising:
at least one microneedle comprising an elongated body extending along a longitudinal axis from a top end to a bottom end and integrally formed on a first side of a substrate, wherein:
the elongated body comprises an upper portion and a lower portion;
the lower portion of the elongated body comprises an internal capillary bore hole extending into the substrate;
the upper portion of the elongated body has a semi-enclosed internal void space formed by at least three body sides whereof two body sides join at a sharp edge and a third body side is provided with an opening slit extending from the lower portion of the elongated body to the upper end of the third body side;
the top end of the elongated body is configured as a bevel to create a sharp tip at the top of said edge, said bevel extending to the third body side;
the semi-enclosed internal void space of the upper portion of the elongated body opening to the internal capillary bore hole of the bottom end of the elongated body; and
the bottom end of the elongated body is connected to the substrate,
wherein each proximal end is integrally formed with the substrate and each capillary bore hole is in fluid communication with an inlet of a fluid channel system according to claim 12, on a second side of the substrate,
wherein the chip is arranged to, upon a microneedle brought in contact with fluid, passively transport said fluid though said microneedle and the fluid channel system to a fluid collection area.

23. A method of fabricating a chip comprising at least one microneedle and a fluid channel according to claim 22 using a micro electro mechanical system fabrication process, the method comprising:
growing a sacrificial oxide layer on a silicon wafer substrate;
masking the sacrificial oxide layer with a patterned photoresist;
removing the sacrificial oxide layer according to the patterned photoresist;
etching the silicon wafer according to the pattern of the removed sacrificial oxide layer using a deep reactive ion etching method;
removing the remaining sacrificial oxide layer by using an etchant.
